(19) United States
(12) Patent Application Publication
(10) Pub. No.: US 2017/0217780 A1
(43) Pub. Date: Aug. 3, 2017

(54) HIMALAYAN SALT CRYSTAL PURIFICATION PROCESS

(71) Applicant: Himalasalt Sustainable Sourcing, LLC, Sheffield, MA (US)

(72) Inventor: Melissa Kushi, Sheffield, MA (US)

(21) Appl. No.: 15/422,164

(22) Filed: Feb. 1, 2017

Related U.S. Application Data
(60) Provisional application No. 62/289,499, filed on Feb. 1, 2016.

(51) Int. Cl. C01D 3/04; B07B 1/00; B07B 9/00; A61K 33/14; B07C 5/342

(52) U.S. Cl. CPC C01D 3/04; A61K 33/14; B07C 5/3425; B07B 9/00; B07B 1/00; C01P 2006/80; C01P 2006/60

(57) ABSTRACT

The disclosure provides a process for obtaining the purest form of Himalayan pink salt. The purest form of Himalayan pink salt is preferably in crystal in form and is substantially clear and colorless. The disclosure provides a purification process that identifies and removes solid impurities affording the purest form of Himalayan pink salt. The system may machine vision algorithms that process feedback from one or more sensors to detect and identify color or clarity, and may use one or more host controllers to remove solid impurities. The controller may identify the color or clarity and ensure consistent removal of the solid impurities.

### RELATED APPLICATION

This application claims the benefit of U.S. Provisional Application No. 62/289,499, filed on Feb. 1, 2016. The entire teachings of the above application are incorporated herein by reference.

### BACKGROUND

Salt is critical for life and is one of the basic elements required by the body. Commercially available table salt, most commonly found in kitchens and restaurants around the world, is highly refined requiring chemical processing to artificially improve the physical properties. With chemical dumping and toxic oil spills polluting the oceans at an alarming rate, most of today's sea salt is not as pure as it used to be. Himalayan pink salt is a naturally occurring, unprocessed raw sea salt mined from salt caves that formed millions of years ago as ocean salt settled into geologic pockets. Since Himalayan pink salt is uncontaminated with toxins or pollutants, the Himalayan pink salt is a healthier alternative that does not burden the body as other sea salts or commercial salts do.

### SUMMARY OF THE DISCLOSURE

There are various grades of Himalayan pink salt. Generally, cloudy, milky or opaque looking crystals represent cheap and widely available Himalayan pink salt that have inconsistent taste qualities. Substantially clear and colorless crystals of Himalayan pink salt represent the rarest and purest grade of Himalayan pink salt which provide the most consistent taste. However, obtaining the purest form of Himalayan pink salt have been limited by high cost, operational complexity, and the inability to efficiently identify and remove undesired impurities. Thus, there is a need for a new and effective process to access the purest form of Himalayan pink salt.

Some example embodiments of the present disclosure provide methods and systems of deriving the purest form of Himalayan pink salt. The purest form of Himalayan pink salt is crystal in form and is substantially clear and colorless.

By way of contrast, cloudy, milky or opaque looking Himalayan pink salt crystals are considered undesirable by the present system/method. Such cloudy/milky/opaque Himalayan pink salt crystals suffer from lack consistency in taste and are widely available.

The purest form of Himalayan pink salts are rare and often difficult to obtain as they are generally limited by high cost, operational complexity, and the inability to efficiently identify and remove undesired impurities.

In one aspect, the disclosure provides example methods and systems configured to evacuate impurities from pure Himalayan pink salt from an array of mesh screens. The purest form of Himalayan pink salt is preferably crystal in form and is substantially clear and colorless. An example method/system may include: contacting an impure mixture of Himalayan pink salt above a mesh screen; vibrating, vacuuming or shaking the screen to allow small particles of impure amorphous solids to pass through the screen leaving a mixture of impure crystal Himalayan pink salts above the mesh screen; identifying and removing colored crystal Himalayan pink salt impurities and colored crystal metal impurities from the mixture of impure crystal Himalayan pink salts until the mixture of impure crystal Himalayan pink salts is substantially clear and colorless; resulting in the purest form of Himalayan pink salt.

In some embodiments, the impure mixture of Himalayan pink salt comprises at least the purest form of Himalayan pink salt, and small particles of impure amorphous solids or a mixture of colored crystal Himalayan pink salt impurities or both.

In some embodiments, the small particles of impure amorphous solids comprises amorphous Himalayan pink salt and amorphous solid metal impurities.

In some embodiments, the amorphous Himalayan pink salt and amorphous solid metal impurities is colored.

In some embodiments, the color is substantially red, orange, pink, white, grey or black.

In some embodiments, the amorphous solid metal impurities comprises potassium salts, calcium salts, magnesium salts, sulfur salts, iron salts, oxide salts, water or a combination thereof.

In some embodiments, the mixture of impure crystal Himalayan pink salt comprises at least the purest form of Himalayan pink salt and colored crystal Himalayan pink salt impurities or colored crystal metal impurities or both.

In some embodiments, the colored crystal Himalayan pink salt impurities comprises potassium salts, calcium salts, magnesium salts, sulfur salts, iron salts, oxide salts, water or a combination thereof.

In some embodiments, the colored crystal metal impurities comprises potassium salts, calcium salts, magnesium salts, sulfur salts, iron salts, oxide salts, water or a combination thereof.

In some embodiments, the colored crystal metal impurities is calcium.

In some embodiments, the colored crystal Himalayan pink salt impurities and colored crystal metal impurities color is substantially red, rose, orange, pink, white, grey or black.

In some embodiments, the colored crystal Himalayan pink salt impurities color is substantially red, rose, orange or pink.

In some embodiments, the color red is selected from the group consisting of scarlet, carmine, ruby, crimson, rose, rusty red, brick red, dark red, maroon, barn red, blood red and dark blood red.

In some embodiments, the color pink is selected from the group consisting of salmon, coral pink, light pink, hot pink, deep pink, champagne pink, pale pink, bright pink and rose pink.

In some embodiments, the color orange is selected from the group consisting of dark orange, peach, apricot, melon, carrot orange, pumpkin, alloy orange, and burnt orange.

In some embodiments, the diameter of the small particles of impure amorphous solids is about 1 μm to about 2000 μm.

In some embodiments, small particles of impure amorphous solids comprises of dust, powder, granule, extra fine grain, fine grain and small grain.

In some embodiments, a computer program product stored on a non-transitory computer readable medium configured to facilitate selecting the purest form of Himalayan pink salt, wherein the purest form of Himalayan pink salt is crystal in form and is substantially clear and colorless, the computer program product including computer readable instructions, which when executed by one or more computer processors, cause the one or more processors to: contacting an impure mixture of Himalayan pink salt above a mesh screen; shaking the screen to allow small particles of impure amorphous solids to pass through the screen leaving a mixture of impure crystal Himalayan pink salts above the mesh screen; extract colored crystal Himalayan pink salt impurities and colored crystal metal impurities from the mixture of impure crystal Himalayan pink salts until the mixture of impure crystal Himalayan pink salts is substantially clear and colorless; resulting in the purest form of Himalayan pink salt.

In some embodiments, one sensor monitors the identity of an impure mixture of Himalayan pink salt above a mesh screen. The sensors may be in communication with a host controller, which is directed the extraction process. In these and other embodiments, other sensors may monitor movement the screen to allow small particles of impure amorphous solids to pass through the screen leaving a mixture of impure crystal Himalayan pink salts above the mesh screen, identifying and removing colored crystal Himalayan pink salt impurities and colored crystal metal impurities from the mixture of impure crystal Himalayan pink salts until the mixture of impure crystal Himalayan pink salts is substantially clear and colorless, resulting in the purest form of Himalayan pink salt.

In some embodiments, the controller may integrate machine vision algorithms (such as object detection processes that are well known in image processing) and predictive algorithms to regulate the identity of an impure mixture of Himalayan pink salt above a mesh screen. Object detection algorithms are well known in the machine vision art and can rely on processes such as Objects look different under varying conditions:

Changes in lighting or color

Changes in viewing direction

Changes in size/shape

See e.g. https://en.wikipedia.org/wiki/Outline_of_object_recognition, the entire teachings of which are incorporated by reference.

In other embodiments, the controller may regulate movement of the screen to allow small particles of impure amorphous solids to pass through the screen leaving a mixture of impure crystal Himalayan pink salts above the mesh screen. In some embodiments, the controller may regulate identifying and removing colored crystal Himalayan pink salt impurities and colored crystal metal impurities from the mixture of impure crystal Himalayan pink salts until the mixture of impure crystal Himalayan pink salts is substantially clear and colorless, resulting in release of the purest form of Himalayan pink salt.

In some embodiments, the one or more sensors maintain an electronic record, such as a log, of one or more of a contact of a solid mixture above a mesh screen, shaking the screen, and the identifying and removing impurities from a solid mixture, and the release of the purest form of Himalayan pink salt.

In some embodiments, a power supply may be linked to a screen or be independent of screen.

In some embodiment's, the purest form of Himalayan pink salt is derived using a host controller, process chamber, array of mesh screens, and a vacuum system. An impure mixture of Himalayan pink salt may be embedded in the array of mesh screens inside a process chamber. A vacuum pump may be coupled to the process chamber.

The vacuum pump may be configured to evacuate small particles of impure amorphous solids from the array of mesh screens by: (i) contacting an impure mixture of Himalayan pink salt in the array of mesh screens; and (ii) vibrating or vacuuming the screen to allow small particles of impure amorphous solids to pass through the screen leaving a mixture of impure crystal Himalayan pink salts above the mesh screen;

Based on feedback from sensors in the process chamber, the host controller may be computationally configured to instruct a robotic arm to identify and remove colored crystal Himalayan pink salt impurities and colored crystal metal impurities from the mixture of impure crystal Himalayan pink salts until the mixture of impure crystal Himalayan pink salts is substantially clear and colorless. In this way, the remaining mixture of impure crystal Himalayan pink salts in the mesh screens is the purest form of Himalayan pink salt.

### BRIEF DESCRIPTION OF THE DRAWINGS

The foregoing will be apparent from the following more particular description of example embodiments of the disclosure, as illustrated in the accompanying drawings in which like reference characters refer to the same parts throughout the different views. The drawings are not necessarily to scale, emphasis instead being placed upon illustrating embodiments of the present disclosure.

FIG. 1 is a flow chart of an example embodiment of the Himalayan pink salt purification process.

FIG. 2A is a schematic diagram of an example computer network environment in which embodiments of the disclosure are deployed.

FIG. 2B is a block diagram of certain components of the computer nodes in the network of FIG. 2A.

FIG. 3 is a schematic diagram of a networked salt purification vacuum system according to an embodiment.

### DETAILED DESCRIPTION OF THE DISCLOSURE

A description of example embodiments of the disclosure follows.

Himalayan pink salt is a naturally occurring, unprocessed raw sea salt mined from salt caves that formed millions of years ago as ocean salt settled into geologic pockets. Since Himalayan pink salt is uncontaminated with toxins or pollutants, the Himalayan pink salt is a healthier alternative that does not burden the body as other sea salts or commercial salts do.

Himalayan pink salt is the perfect economical companion for any refillable fine shaker and refillable coarse grinder.

As there are differing grades of chocolate, coffee, and tea, there is also differing grades of Himalayan salt. Only substantially clear and colorless crystal Himalayan salts are considered super premium grade crystals and is the rarest form of crystalline clear Halite. Substantially clear and colorless crystal Himalayan salts are known truly as the diamond of salt. When added together with rich Himalayan salt hues of rose, pink, and orange crystal, Himalayan pink salts can provide the perfect balance of flavor and healthy essential minerals.

Only the purest form of Himalayan pink salt use the strictest criteria for screening Himalayan salt. The higher the clarity, the more rare the grade of Himalayan salt. Unlike cloudy, milky, opaque or dirty looking salt, which is cheap and plentiful, clear and colorless diamond like crystals are created through the highest levels of geological compression and are the most rare and sought salt for flavor and health.

Clear Himalayan salt of roses, pinks and oranges are superior to the darker reds and cloudy pinks flooding the market. Generally, the darker colored salts taste bitter. Thus, cheap inferior grades of Himalayan salt are undesirable. The darker crystals are found in surface veins and contain more insoluble matter, making it difficult to assimilate. Not only can a visual difference can be seen in the purest form of Himalayan pink salt, a difference in taste can also be noticed.

Despite advances in mining for Himalayan pink salt, obtaining the purest form of Himalayan pink salt have been limited by high cost, operational complexity, and the inability to efficiently identify and remove undesired impurities. Therefore, there is a need for new and effective processes to access the purest form of Himalayan pink salt.

The present disclosure provides a novel method of selecting the purest form of Himalayan pink salt, wherein the purest form of Himalayan pink salt is crystal in form and is substantially clear and colorless. The disclosure provides identifying and removing Himalayan pink salt impurities affording the purest form of Himalayan pink salt. As described herein, the method comprising: contacting an impure mixture of Himalayan pink salt above a mesh screen; shaking the screen to allow small particles of impure amorphous solids to pass through the screen leaving a mixture of impure crystal Himalayan pink salts above the mesh screen; identifying and removing colored crystal Himalayan pink salt impurities and colored crystal metal impurities from the mixture of impure crystal Himalayan pink salts until the mixture of impure crystal Himalayan pink salts is substantially clear and colorless; resulting in the purest form of Himalayan pink salt.

The present disclosure relates to a computer program product stored on a non-transitory computer readable medium configured to facilitate selecting the purest form of Himalayan pink salt, wherein the purest form of Himalayan pink salt is crystal in form and is substantially clear and colorless, the computer program product including computer readable instructions, which when executed by one or more computer processors, cause the one or more processors to: contacting an impure mixture of Himalayan pink salt above a mesh screen; vibrating or vacuuming the screen to allow small particles of impure amorphous solids to pass through the screen leaving a mixture of impure crystal Himalayan pink salts above the mesh screen; identifying and removing colored crystal Himalayan pink salt impurities and colored crystal metal impurities from the mixture of impure crystal Himalayan pink salts until the mixture of impure crystal Himalayan pink salts is substantially clear and colorless; resulting in the purest form of Himalayan pink salt.

### I. Example Definitions

As used herein, a “mesh screen” means any knit, woven, or knotted material of open texture. A mesh screen can be any interwoven or intertwined structure with open space between the strands of network. The open spaces can range from 1 μm to 5000 μm.

As used herein, “shaking” can be accomplished manually or mechanically.

As used herein, “impure mixture of Himalayan pink salt” means a mixture substantially comprising pure forms of Himalayan pink salts, mixtures of impure crystal Himalayan pink salts and small particles of impure amorphous solids.

As used herein, “mixtures of impure crystal Himalayan pink salts” means a mixture substantially comprising pure forms of Himalayan pink salts, colored crystal Himalayan pink salt impurities and colored crystal metal impurities.

As used herein, “small particles of impure amorphous solids” means a mixture substantially comprising amorphous Himalayan pink salt and amorphous solid metal impurities.

As used herein, “solid metal impurities” means a mixture substantially comprising potassium salts, calcium salts, magnesium salts, sulfur salts, iron salts, oxide salts, water or a combination thereof.

As used herein, “colored crystal Himalayan pink salt impurities” means a mixture substantially comprising potassium salts, calcium salts, magnesium salts, sulfur salts, iron salts, oxide salts, water or a combination thereof. Colored crystal Himalayan pink salt impurity color can be substantially red, orange, pink, white, grey or black. Colored crystal Himalayan pink salt impurity color can also be substantially red, rose, orange or pink.

As used herein, “colored crystal metal impurities” means a mixture substantially comprising potassium salts, calcium salts, magnesium salts, sulfur salts, iron salts, oxide salts, water or a combination thereof. Colored crystal metal impurity color can be substantially red, rose, orange, pink, white, grey or black.

As used herein, “purest” means substantially pure.

As used herein, “the color red” can be scarlet, carmine, ruby, crimson, rose, rusty red, brick red, dark red, maroon, barn red, blood red and dark blood red.

As used herein, “the color pink” can be salmon, coral pink, light pink, hot pink, deep pink, champagne pink, pale pink, bright pink and rose pink.

As used herein, “the color orange” can be dark orange, peach, apricot, melon, carrot orange, pumpkin, alloy orange, and burnt orange.

As used herein, “diameter” can range from about 0.1 μm to about 5000 μm.

As used herein, “small particles” means dust, powder, granule, flakes, extra fine grain, fine grain and small grain.

As used herein, “halite” means a color or colorless mineral occurring in cubic crystals with perfect cleavage. Representative examples of halites are NaCl, Na₂SO₄, K₂Ca₂Mg(SO₄)₄.2H₂O, FeO, Fe₃O₄, Fe₂O₃, Fe(OH)₂ and Fe(OH)₃.

### II. Himalayan Pink Salt Purification Process

The following examples serve to illustrate, and in no way limit the present disclosure.

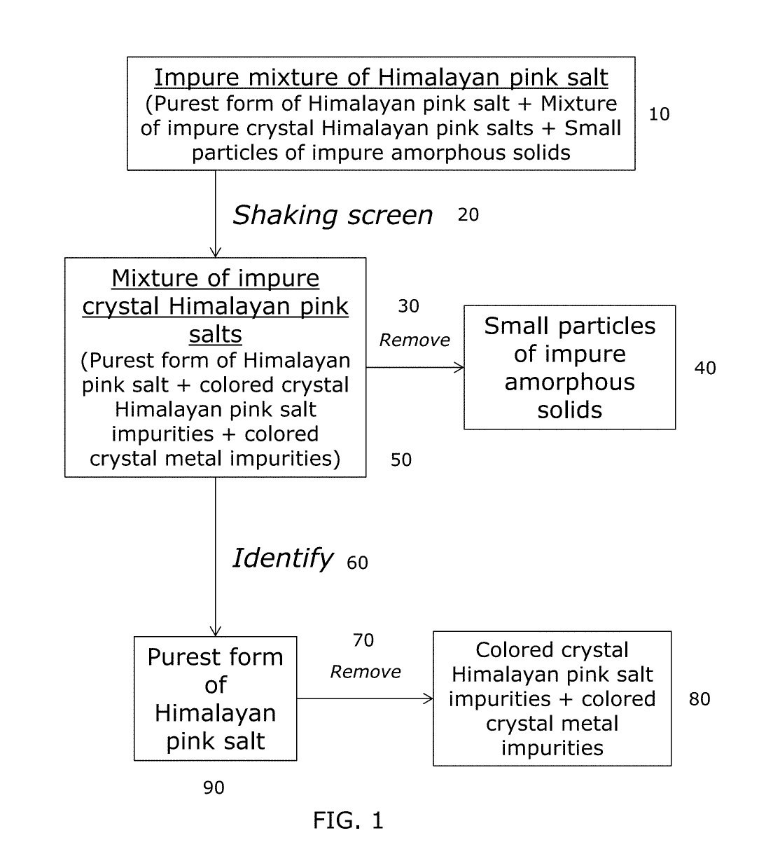

An example implementation of the Himalayan pink salt purification process is depicted in FIG. 1. An impure mixture of Himalayan pink salt 10 comprising pure forms of Himalayan pink salt, mixtures of impure crystal Himalayan pink salts and small particles of impure amorphous solids is placed on top of a mesh screen. The mesh screen can have open spaces that can range from 1 μm to 5000 μm, but preferably range from 2000 μm to 5000 μm. The screen can be shaken 20 to allow separation 30 of the small particles of impure amorphous solids 40 by falling below the mesh screen affording a mixture of impure crystal Himalayan pink salt 50. The screen is gently shaken 20 to prevent damage to the remaining material (mixture of impure crystal Himalayan pink salts 50) on top of the screen. The mixture of impure crystal Himalayan pink salt 50 that remains on top of the screen comprises pure forms of Himalayan pink salts, colored crystal Himalayan pink salt impurities and colored crystal metal impurities. The mixture of impure crystal Himalayan pink salt 50 is then inspected visually to identify 60 colored crystal Himalayan pink salt impurities and colored crystal metal impurities. The identified colored solids (colored crystal Himalayan pink salt impurities and colored crystal metal impurities) 80 are then removed 70 to afford a clear and colorless crystal that represents the purest form of Himalayan pink salt 90.

### III. Digital Processing Environment

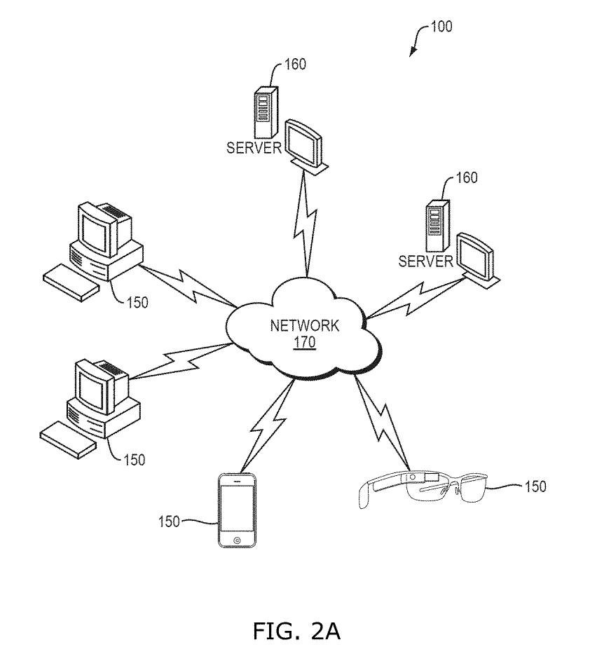

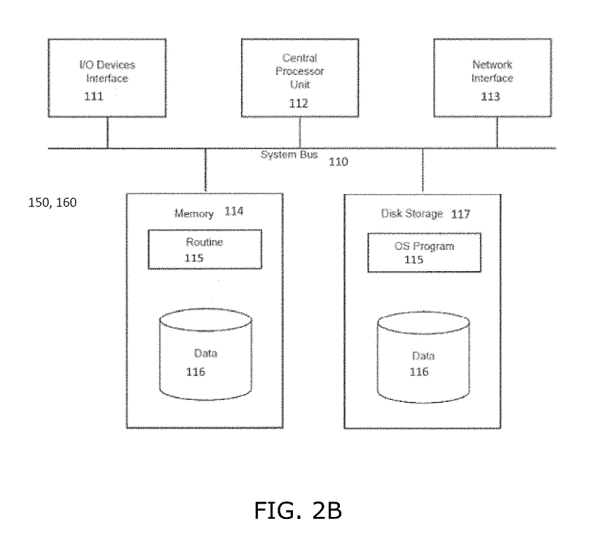

Example implementations of controllers to purify Himalayan pink salt may be implemented in a software, firmware, or hardware environment. FIG. 2A illustrates one such example digital processing environment in which embodiments of the present disclosure may be implemented. Client computers/devices 150 and server computer/devices 160 (or a cloud network 170) provide processing, storage, and input/output devices executing application programs and the like.

Client computers/devices 150 may be linked directly or through communications network 170 to other computing devices, including other client computers/devices 150 and server computer/devices 160. The communication network 170 can be part of a wireless or wired network, remote access network, a global network (i.e. Internet), a worldwide collection of computers, local area or wide area networks, and gateways, routers, and switches that currently use a variety of protocols (e.g. TCP/IP, Bluetooth®, RTM, etc.) to communicate with one another. The communication network 170 may take a variety of forms, including, but not limited to, a data network, voice network (e.g. land-line, mobile, etc.), audio network, video network, satellite network, radio network, and pager network. Other electronic device/computer networks architectures are also suitable.

Client computers/devices 150 may be sensors, such as color and clarity sensors, that monitor and log conditions on top of the screen. Server computers 160 may be controllers configured to provide a controlled environment system 100 which communicates with client devices 150, such as the color and clarity sensors, for controlling the removal of impure solids. The server computers may not be separate server computers but part of cloud network 170. In some embodiments, a server computer (controller) may operate locally within the purification process. In these embodiment and other embodiments, the controller may be an industrial programmable logic controller (PLC), or other such controller configured with firmware similar to PLC firmware. The sensors 150 may communicate information regarding the solid mixtures, such as the color and clarity of the solids, to the controllers 160. In some embodiments, the sensors 150 may include client applications executing on the sensors 150 for monitoring and logging the conditions, and communicating the information regarding the conditions to the controllers 160. Client computers/devices 150 may also be devices to configure the sensors 150 and controllers 160, such as configuring the color and clarity thresholds for identifying and removing solid impurities.

FIG. 2B is a block diagram of any internal structure of a computer/computing node (e.g., client processor/device 150 or server computers 160) in the processing environment of FIG. 2A, which may be used to facilitate processing audio, image, video or data signal information. Each computer 150, 160 in FIG. 2B contains a system bus 110, where a bus is a set of actual or virtual hardware lines used for data transfer among the components of a computer or processing system. The system bus 110 is essentially a shared conduit that connects different elements of a computer system (e.g., processor, disk storage, memory, input/output ports, etc.) that enables the transfer of data between elements.

Attached to the system bus 110 is an I/O device interface 111 for connecting various input and output devices (e.g., keyboard, mouse, touch screen interface, displays, printers, speakers, audio inputs and outputs, video inputs and outputs, microphone jacks, etc.) to the computer 150, 160. A network interface 113 allows the computer to connect to various other devices attached to a network (for example the network illustrated at 170 of FIG. 2A). Memory 114 provides volatile storage for computer software instructions 115 and data 116 used to implement software implementations of the present disclosure.

Software components 115, 116 of the controlled environment system 100 (e.g. FIGS. 2A and 2B) described herein may be configured using any programming language, including any high-level, object-oriented programming language. The system may include other instances of client processes executing on other client computers/devices 150, such as a client application that may communicate with the server (e.g., controller) to configure the parameters for removing solid impurities from the screen. In some embodiments, the computing device 150 for configuring the parameters may be implemented via a software embodiment and may operate, at least partially, within a browser session.

In an example mobile implementation, a mobile agent implementation of the disclosure may be provided. A client server environment can be used to enable mobile configuration or monitoring of the sensors 150 or controller 160. It can use, for example, the XMPP protocol to tether a configuration server 115 on a device 150 to controller 160 or sensor 150. The server 160 can then issue commands via the mobile phone on request. The mobile user interface framework to access certain components of the system 100 may be based on XHP, Javelin and WURFL. In another example mobile implementation for OS X, iOS, and Android operating systems and their respective APIs, Cocoa and Cocoa Touch may be used to implement the client side components 115 using Objective-C or any other high-level programming language that adds Smalltalk-style messaging to the C programming language.

Disk storage 117 provides non-volatile storage for computer software instructions 115 (equivalently “OS program”) and data 116 used to implement embodiments of the system 100. The system may include disk storage accessible to the server computer 160. The server computer (e.g., controller) or client computer (e.g., sensors) may store information, such as logs, regarding the color or clarity of the solid impurities. Central processor unit 112 is also attached to the system bus 110 and provides for the execution of computer instructions. Software implementations 115, 116 may be implemented as a computer readable medium capable of being stored on a storage device 117, which provides at least a portion of the software instructions for the purification process. Executing instances of respective software components of the purification process, may be implemented as computer program products 115 (e.g., PLC firmware), and can be installed by any suitable software installation procedure, as is well known in the art. In another embodiment, at least a portion of the system software instructions 115 may be downloaded over a cable, communication and/or wireless connection via, for example, a browser SSL session or through an app (whether executed from a mobile or other computing device). In other embodiments, the system 100 software components 115, may be implemented as a computer program propagated signal product embodied on a propagated signal on a propagation medium (e.g. a radio wave, an infrared wave, a laser wave, a sound wave, or an electrical wave) propagated over a global network such as the Internet, or other networks. Such carrier medium or signal provides at least a portion of the software instructions for the present controlled environment system 100 of FIG. 2A.

### IV. Sea Salt Crystals Purification Vacuum System

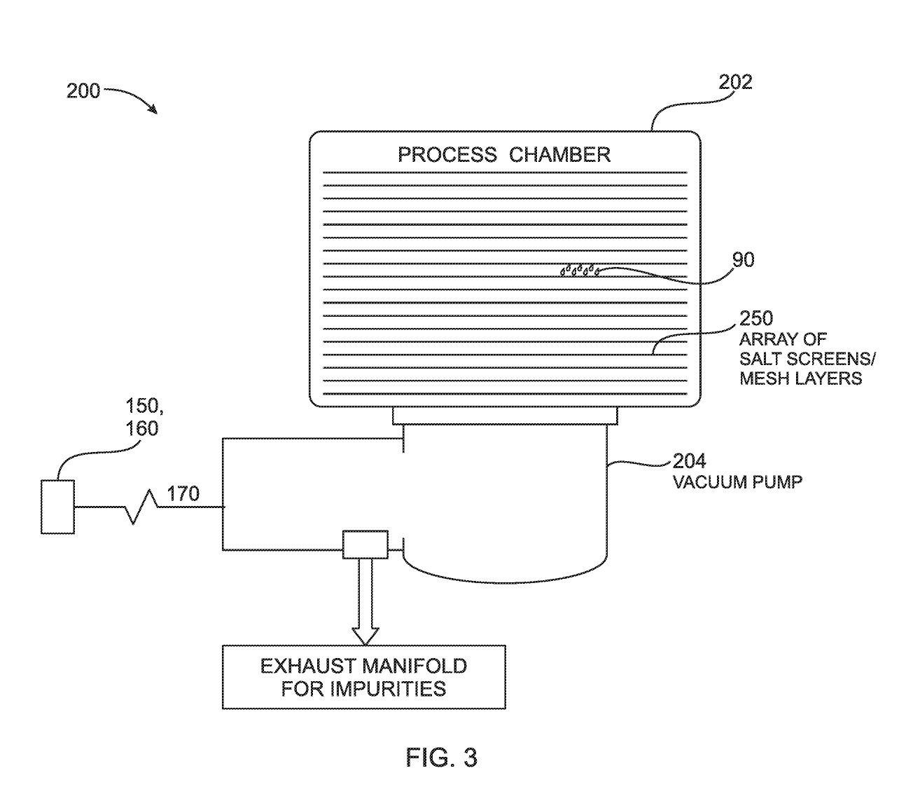

FIG. 3 is a diagram of a sea salt purification vacuum system 200 according to an embodiment. The sea salt purification vacuum system 200 is coupled to process chamber 202. The vacuum system evacuates impure salt crystals from the process chamber 202. For instance, small particles of impure amorphous solids comprises of dust, powder, granule, extra fine grain, fine grain and small grain may be evacuated from the process chamber 202 by creating a vacuum. The vacuum system 200 includes at least one vacuum pump 204 to create a vacuum in the chamber 202. The vacuum pump 204 may be a roughing pump, getter pump, turbopump, water pump, dehumidifier pump, or cryopump. See, e.g. https://en.wikipedia.org/wiki/Vacuum_pump, the entire teachings of which are incorporated by reference.

The vacuum pump may be used to remove trace elements of impurities from the salt crystals including small particles of impure amorphous solids. For example, an array of screens and mesh layers may be stacked in the process chamber 202 in which the salt crystals are sandwiched. A vacuum may be created in the process chamber using the vacuum pump 204. This can allow separation of the small particles of impure amorphous solids by creating a vacuum within the process chamber such that impurities are exhausted into an exhaust manifold. A dehumidifier pump may be used to remove moisture from the salt crystals. A cryopump may be used to remove any high boiling point gasses from the salt crystals. The remaining molecules on the screen/mesh array 250 affording a mixture of impure crystal Himalayan pink salt.

Each of the screens and/or mesh layers in the array 250 may be positioned in close proximity. In this way, the compact positioning can substantially prevent movement and damage to pure crystal Himalayan pink salts 90 during the vacuum process. After the vacuum cycle, the pure crystal Himalayan pink salt crystals would remain on the screens and mesh layers 240. The strength of the vacuum may be adjusted by a variable speed drive motor of the vacuum 204 to protect the pure crystal Himalayan pink salts from damage, such that the speed of the drive motor of the vacuum is varied or cycled on and off.

The crystal Himalayan pink salt that remains on the screen array 250 comprises pure forms of Himalayan pink salts 90. Any colored crystal metal impurities that may remain may be identified and or removed with a magnetic extraction process or metal detection process. Magnets and metal detectors are well known in the art. In one embodiment, a host control system 150, 160 may be used to instruct a robotic arm to remove the remaining impurities. The remaining substance in the screen/mesh array is a clear and colorless crystal substrate that represents the purest form of Himalayan pink salt 90.

Preferably, a standardized communication link 170 from the vacuum system 204 to a host control system 150, 160. Control of the pump's local electronics is preferably fully integrated with the host control system 150, 160. In this way, the host control system controls the purification process remotely and may be instructed by the purification process software 115.

While this invention has been particularly shown and described with references to example embodiments thereof, it will be understood by those skilled in the art that various changes in form and details may be made therein without departing from the scope of the invention encompassed by the appended claims.

What is claimed is:

1. A method of deriving a purest form of Himalayan pink salt, wherein the purest form of Himalayan pink salt is crystal in form and is substantially clear and colorless, the method comprising:
evacuating small particles of impure amorphous solids from an array of mesh screens by:
contacting an impure mixture of Himalayan pink salt in the array of mesh screens;
vibrating or vacuuming the screen to allow small particles of impure amorphous solids to pass through the screen leaving a mixture of impure crystal Himalayan pink salts above the mesh screen;
identifying and removing colored crystal Himalayan pink salt impurities and colored crystal metal impurities from the mixture of impure crystal Himalayan pink salts until the mixture of impure crystal Himalayan pink salts is substantially clear and colorless; and
providing the remainder on the array of mesh screens, the remainder being the mixture of impure crystal Himalayan pink salts from the array of mesh screens, where the mixture of impure crystal Himalayan pink salts from the array of mesh screens is the purest form of Himalayan pink salt.

2. The method of claim 1, wherein the vibrating or vacuuming is implemented using a vacuum pump.

3. The method of claim 2, wherein the vacuum pump is coupled to a process chamber housing the array of mesh screens.

4. The method of claim 1, wherein the impure mixture of Himalayan pink salt comprises at least the purest form of Himalayan pink salt, and small particles of impure amorphous solids or a mixture of colored crystal Himalayan pink salt impurities or both.

5. The method of claim 4, wherein the small particles of impure amorphous solids comprises amorphous Himalayan pink salt and amorphous solid metal impurities.

6. The method of claim 5, wherein the amorphous Himalayan pink salt and amorphous solid metal impurities is colored, such that the color is substantially red, orange, pink, white, grey or black.

7. The method of claim 6, wherein the amorphous solid metal impurities comprises potassium salts, calcium salts, magnesium salts, sulfur salts, iron salts, oxide salts, water or a combination thereof.

8. The method of claim 1, wherein the mixture of impure crystal Himalayan pink salt comprises at least the purest form of Himalayan pink salt and colored crystal Himalayan pink salt impurities or colored crystal metal impurities or both.

9. The method of claim 8, wherein the colored crystal Himalayan pink salt impurities comprises potassium salts, calcium salts, magnesium salts, sulfur salts, iron salts, oxide salts, water or a combination thereof.

10. The method of claim 8, wherein the colored crystal metal impurities comprises potassium salts, calcium salts, magnesium salts, sulfur salts, iron salts, oxide salts, water or a combination thereof.

11. The method of claim 10, wherein the colored crystal metal impurities is calcium.

12. The method of claim 8, wherein the colored crystal Himalayan pink salt impurities and colored crystal metal impurities color is substantially red, rose, orange, pink, white, grey or black, such that the colored crystal Himalayan pink salt impurities color is substantially red, rose, orange or pink.

13. The method of claim 8, wherein the color red is selected from the group consisting of scarlet, carmine, ruby, crimson, rose, rusty red, brick red, dark red, maroon, barn red, blood red and dark blood red.

14. The method of claim 8, wherein the color pink is selected from the group consisting of salmon, coral pink, light pink, hot pink, deep pink, champagne pink, pale pink, bright pink and rose pink, such that the color orange is selected from the group consisting of dark orange, peach, apricot, melon, carrot orange, pumpkin, alloy orange, and burnt orange.

15. The method of claim 2, wherein the diameter of the small particles of impure amorphous solids evacuated from the array of mesh screens is about 1 μm to about 2000 μm, such that the impure amorphous solids are evacuated into an exhaust manifold coupled to the vacuum pump.

16. The method of claim 1, wherein the small particles of impure amorphous solids comprises of dust, powder, granule, extra fine grain, fine grain and small grain, the small particles of impure amorphous solids being small relative to the mixture of impure crystal Himalayan pink salts.

17. The method of claim 1 wherein a host controller computer system in communication with the vacuum pump instructs the evacuating of the small particles of impure amorphous solids from an array of mesh screens by instructing a drive motor of the vacuum pump to drive the vacuum pump at a variable rate to protect the mixture of impure crystal Himalayan pink salts from excessive vibration and resulting damage.

18. The method of claim 1 wherein the array of mesh screens is arranged in a substantially compact configuration to protect the mixture of impure crystal Himalayan pink salts from excessive vibration and resulting damage during the vacuuming or vibrating.

19. A computer program product stored on a non-transitory computer readable medium configured to derive the purest form of Himalayan pink salt, wherein the purest form of Himalayan pink salt is crystal in form and is substantially clear and colorless, the computer program product including computer readable instructions, which when executed by one or more computer processors, cause the one or more processors to:
evacuating small particles of impure amorphous solids from an array of mesh screens by:
contacting an impure mixture of Himalayan pink salt in the array of mesh screens;
vibrating or vacuuming the screen to allow small particles of impure amorphous solids to pass through the screen leaving a mixture of impure crystal Himalayan pink salts above the mesh screen;
identifying and removing colored crystal Himalayan pink salt impurities and colored crystal metal impurities from the mixture of impure crystal Himalayan pink salts until the mixture of impure crystal Himalayan pink salts is substantially clear and colorless; and
extracting the mixture of impure crystal Himalayan pink salts from the array of mesh screens, where the mixture of impure crystal Himalayan pink salts from the array of mesh screens is the purest form of Himalayan pink salt.

20. A system configured to derive the purest form of Himalayan pink salt, the system comprising:
a process chamber providing an impure mixture of Himalayan pink salt embedded in the array of mesh screens;
a vacuum pump, coupled to the process chamber, configured to evacuating small particles of impure amorphous solids from an array of mesh screens by:
contacting an impure mixture of Himalayan pink salt in the array of mesh screens; and
vibrating or vacuuming the screen to allow small particles of impure amorphous solids to pass through the screen leaving a mixture of impure crystal Himalayan pink salts above the mesh screen;
a host controller computationally instructing a robotic arm to identify and remove colored crystal Himalayan pink salt impurities and colored crystal metal impurities from the mixture of impure crystal Himalayan pink salts until the mixture of impure crystal Himalayan pink salts is substantially clear and colorless; and
where the mixture of impure crystal Himalayan pink salts remaining in the array of mesh screens is the purest form of Himalayan pink salt.
